(19) United States
(12) Patent Application Publication
(10) Pub. No.: US 2025/0236584 A1
(43) Pub. Date: Jul. 24, 2025

(54) IMPROVED METHOD FOR PRODUCING HIGH-PURITY BUTYL ACRYLATE

(71) Applicant: ARKEMA FRANCE, COLOMBES (FR)

(72) Inventors: Serge TRETJAK, SAINT AVOLD CEDEX (FR); Camille HILPERT, SAINT AVOLD CEDEX (FR)

(73) Assignee: ARKEMA FRANCE, COLOMBES (FR)

(21) Appl. No.: 18/703,495

(22) PCT Filed: Oct. 18, 2022
(86) PCT No.: PCT/FR2022/051965

§ 371 Date: Apr. 22, 2024

(30) Foreign Application Priority Data
Oct. 25, 2021 (FR) FR2111319

(51) Int. Cl. C07C 67/08; C07C 67/54

(52) U.S. Cl. CPC C07C 67/08; C07C 67/54

(57) ABSTRACT

The present invention concerns the production of butyl acrylate by direct esterification of acrylic acid with butanol, this reaction being catalyzed by sulfuric acid. More specifically, it relates to an improved process for producing butyl acrylate, comprising a step of upgrading the heavy by-products generated during this production, leading to a high yield of a product meeting the standards in terms of purity and acidity, under optimized energy conditions.

### TECHNICAL FIELD

The present invention concerns the production of butyl acrylate by direct esterification of acrylic acid with butanol, this reaction being catalyzed by sulfuric acid. More specifically, it relates to an improved process for producing butyl acrylate, comprising a step of upgrading the heavy by-products generated during this production, leading to a high yield of a product meeting the standards in terms of purity and acidity, under optimized energy conditions.

### TECHNICAL BACKGROUND AND TECHNICAL PROBLEM

The esterification of acrylic acid is a balanced reaction with generation of water that must be removed during the reaction to shift the equilibrium in the direction of production of the acrylic ester.

The problems that arise in the production of butyl acrylate by direct esterification of acrylic acid, generally in the presence of sulfuric acid as catalyst, are most often related to the complexity of the purification steps necessary after the reaction step to obtain a product of high-purity, to the detriment of the yield of the process.

The industrial process, as described in patent EP 0609127 of the applicant, consists in esterifying acrylic acid (AA) with excess butanol, in the presence of sulfuric acid. The reaction mixture at the end of reaction comprises butyl acrylate, residual acrylic acid, butyl hydrogen sulfate, traces of sulfuric acid and various impurities resulting from side reactions. This reaction mixture is then subjected to a step of neutralization and washing with water, the object of which is to remove the so-called acidic impurities: residual sulfuric acid, butanol hydrogen sulfate and acrylic acid. This mixture, free of acidic impurities, undergoes various purification steps, which lead to the upgrading of the purified butyl acrylate. One of the steps, referred to as topping, consists in particular in distilling the butanol and the light by-products. Butanol can thus be recycled to the esterification reaction.

The final step of purification of butyl acrylate consists in sending the mixture containing the ester from which the light products have been removed to a last distillation column from which it exits at the top, purified of the heavy by-products which are found at the bottom of the distillation column and are then concentrated in an evaporator. The top product of this evaporator, namely butyl acrylate (BuA), is returned to the bottom of the rectification column, which makes it possible, on the one hand, to upgrade it as a finished product but also to keep the temperature of the bottom of the column low enough to avoid fouling problems related to the heat-sensitive nature of this monomer.

The evaporator residue, in addition to the Michael derivatives and a few percent of free monomers, and also a few percent of polymerization inhibitors, accumulated during all of the purification steps, such as mainly phenothiazine in its free form or as an adduct of AA or BuA, and also heavy compounds of polymeric nature which are more or less soluble in the medium. In general, this residue is eliminated by incineration, which leads to a significant loss of yield.

Among the by-products generated according to the side reactions, mention may be made of light products such as butyl acetate, butyl propionate, dibutyl ether, isobutyl acrylate or heavy products such as dibutyl maleate.

“Heavy” compounds resulting from Michael addition reactions spontaneously form in the butyl acrylate production units. These parasitic reactions are promoted by high temperatures encountered in particular in the distillation column bottoms of these units.

Thus acrylic acid, unreacted butanol or water of reaction are added to the double bond of the butyl acrylate to form mainly:
butyl acryloxypropionate (AA/BuA) by addition of acrylic acid (AA) to butyl acrylate (BuA);
butyl hydroxypropionate (BHP) by addition of water to butyl acrylate;
butyl butoxypropionate (BBP) by addition of butanol to butyl acrylate.

Polyaddition or the formation of mixed compounds is also possible.

One of the characteristics of heavy by-products is that their boiling point is above the boiling points of acrylic acid, butanol and butyl acrylate. Since their volatility is low, they accumulate at the bottom of the last distillation column, at the bottom of the evaporator used to concentrate this residue.

Various solutions have been proposed for the upgrading of these heavy by-products still containing polymerization inhibitors.

Document CN1063678 proposes methods of treating the oxy-esters formed during the synthesis of butyl acrylate using protic acid catalysts such as sulfuric acid or para-toluenesulfonic acid. Compounds such as phthalates can also be added, as described in document U.S. Pat. No. 4,293,347.

The disadvantage of these cracking methods is that the residual product is viscous and contains solids. U.S. Pat. No. 6,617,470 proposes the use, as catalysts, of arylsulfonic acids such as dodecylsulfonic acid, which suppresses the formation of solids in the bottom residue.

Document US 2011/0230675 proposes adding water continuously when cracking is carried out by acid catalysis, in order to avoid the formation of solid plate-out.

Finally, document CN102173990 proposes adding copper salts to the cracker feed in order to facilitate the subsequent treatment of the ultimate cracking residue.

Document U.S. Pat. No. 5,767,306 describes a process for producing butyl acrylate by direct esterification of AA with butanol in the presence of sulfuric acid as catalyst and of at least one polymerization inhibitor, comprising distillation steps for separating a stream containing heavy by-products which is concentrated on a falling stream evaporator generating two streams: a stream for feeding a cracker and a stream containing a residue with the polymerization inhibitors.

The applicant company has described in its application FR2101402 a process diagram combining a side draw column, a thermal or thermal and catalytic cracker and a decanter and washing system for treating the products obtained from the cracker for the synthesis of acrylic ester such as 2-ethylhexyl acrylate. This combination is made necessary because, in these processes catalyzed by acid resins, the so-called acid impurities are not, on the one hand, removed during a neutralization operation prior to the purification section and, on the other hand, are also generated during cracking. Without the combination of side draw column—decanter (see example 2), it is not possible to obtain a purified 2-ethylhexyl acrylate meeting the specifications.

Without the applicant being bound to any explanation, it believes that it is the interaction between phenothiazine and the acid catalyst during catalytic and thermal cracking that is largely responsible for the formation of solid in the ultimate residue of the cracker.

Application FR2108885 uses a thermal cracker without catalyst, which makes it possible to eliminate this interaction successfully. However, the cracking yield remains lower than that obtained during thermal and catalytic cracking.

To solve the problem of the clogging of equipment, patent FR 2901272 implements a vacuum distillation operation at a relatively low temperature, under an inert atmosphere, optionally in the presence of a dispersant, in order to limit the phenothiazine content in the distillation product to a value of less than 100 ppm, before carrying out thermal and catalytic cracking at atmospheric pressure under an inert atmosphere.

In this process, the phenothiazine removal operation requires a boiler, a distillation column operated at reduced pressure, a condenser, reflux equipment and a devesiculator and an injection of an inert gas. The combination of this distillation to remove phenothiazine and of catalytic cracking at atmospheric pressure, in the presence of an inert gas, avoids the formation of solids in the residue with a very satisfactory cracking rate. Thus, the process as envisioned comprises a rectification column, an evaporator at the bottom of this column, distillation under reduced pressure under inert gas and finally thermal and catalytic cracking under inert gas as well.

There remains a need to simplify this process scheme, which remains laborious to implement.

It has now been found that by replacing the evaporator at the bottom of the rectification column and the distillation under reduced pressure and under inert atmosphere with a single evaporator and its staged condensation system, the formation of solid in the residue of the cracker is significantly reduced, while simplifying the equipment used, in a process allowing the production of high-purity butyl acrylate.

### SUMMARY OF THE INVENTION

The present invention describes an evaporation system which makes it possible, by recycling butyl acrylate, to maintain a rectification column bottom temperature compatible with the heat-sensitive nature of butyl acrylate and to send the Michael adducts to the thermal and catalytic cracker, while purging the very heavy compounds and polymerization inhibitors at the bottom of this evaporator, for a process allowing the production of high-purity butyl acrylate, as described in patent EP 0609127 for the reaction part.

It also adds to the purification scheme described in this patent by combining an evaporator placed at the bottom of the butyl acrylate purification column with its staged condensation system and describes the recycling of the top products from the cracking into the process.

The invention relates to a process for producing butyl acrylate by direct esterification of acrylic acid with excess butanol in the presence of sulfuric acid as catalyst and at least one polymerization inhibitor, resulting in production of a crude reaction mixture containing butyl acrylate, residual acrylic acid and residual butanol, butyl hydrogen sulfate, traces of sulfuric acid and impurities resulting from side reactions, said process comprising neutralization and washing steps with water leading to the production of a reaction mixture free of so-called acidic impurities, characterized in that said reaction mixture washed of acidic impurities is subjected at least to the following steps i), ii) and iii):
i) topping in a distillation column to obtain:
   at the top, a stream composed essentially of unreacted reactants;
   at the bottom, a stream comprising the desired ester and heavy by-products;
ii) the bottom stream from the topping column is subjected to a rectification column to separate:
   at the top, the purified desired ester;
   at the bottom, a stream containing heavy by-products, which is concentrated on a thin-film evaporator with a cooled top stream in two steps in order to recycle the light compounds present to the rectification column, to remove a final residue containing the polymerization inhibitor(s) and to send the Michael adducts with a reduced content of inhibitor(s), in particular phenothiazine, at less than 1000 ppm, preferably less than 300 ppm, to the cracker;
(iii) the stream containing the Michael adducts that are sent to a thermal and catalytic cracker to obtain:
   at the top, the noble products resulting from the cracking which are recycled to the reaction;
   at the bottom, the ultimate residue that is sent to a waste treatment plant.

The present invention makes it possible to overcome the disadvantages of the state of the art. More particularly, it provides a process for obtaining a high-purity butyl acrylate having as specifications an ester purity greater than 99.5%, integrating an optimized process for removing the polymerization inhibitors, making it possible to crack the Michael adducts into reactants (acrylic acid and alcohol) and into finished product, thus increasing the yield of the process by limiting the amount of residue to be removed.

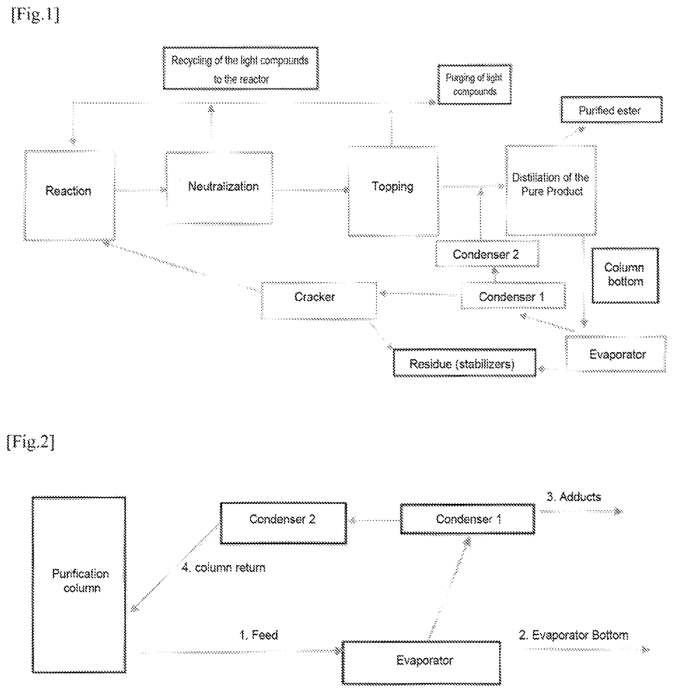

Other characteristics and advantages of the invention will emerge more clearly on reading the detailed description which follows, with reference to appended FIGS. 1 and 2.

FIG. 1: overall diagram of the process for synthesis of butyl acrylate according to the invention, with an exchanger at the bottom of the butyl acrylate purification column.

FIG. 2: evaporation process according to the invention with its staged condensation system.

### DETAILED DESCRIPTION OF THE INVENTION

The invention relates to a process for producing butyl acrylate by direct esterification of acrylic acid with excess butanol, in the presence of sulfuric acid as catalyst, and at least one polymerization inhibitor, resulting in production of a crude reaction mixture containing butyl acrylate, residual acrylic acid and residual butanol, butyl hydrogen sulfate, traces of sulfuric acid and impurities resulting from side reactions.

According to various implementations, said process comprises the following features, where appropriate in combination.

After the esterification step, the process according to the invention comprises neutralization and washing steps with water leading to the production of a reaction mixture freed of the so-called acidic impurities: sulfuric acid, butyl hydrogen sulfate, acrylic acid dimer and residual acrylic acid.

Typically, the reaction mixture washed of acidic impurities as described above is subjected to at least the following steps i), ii) and iii):
i) topping in a distillation column to obtain:
   at the top, a stream composed essentially of unreacted reactants;
   at the bottom, a stream comprising the desired ester and heavy by-products;
ii) the bottom stream from the topping column is subjected to a rectification column to separate:
   at the top, the purified desired ester;
   at the bottom, a stream containing heavy by-products and the inhibitors present in a content of the order from 1% to 3% in the preceding purification steps, which is concentrated on a thin-film evaporator generating three streams:
   a butyl acrylate stream to be recycled to the rectification column;
   a Michael adduct stream for feeding the cracker;
   a stream containing residue containing the polymerization inhibitors;
(iii) the Michael adduct stream is subjected to thermal and catalytic treatment performed in a cracker to separate:
   at the top, a stream of upgradable products which is recycled to the reaction;
   at the bottom, a residue sent to a waste treatment plant.

Reaction temperature and pressure of the evaporator are linked so that the butyl acrylate and the Michael adducts are evaporated off.

According to one embodiment, the evaporation is carried out in a temperature range of 80° C. to 100° C. and more especially between 90° C. and 100° C.

According to one embodiment, the pressure maintained in the evaporator is between 800 Pa and 2000 Pa.

According to one embodiment, the stream at the top of this evaporator is cooled in two successive steps:
a partial condensation in a temperature range of from 50 to 80° C. and more especially 60° C.-70° C. at the same pressure as that of the evaporator to obtain, on the one hand, a liquid stream of Michael adducts, having a reduced content of inhibitors of less than 1000 ppm, going to feed the cracker, and, on the other hand, a vapor stream, and
a condensation of this vapor stream in a temperature range of from 20° C. to 40° C. and more especially 20° C.-30° C. before taking up this liquid stream via a pump and feeding the rectification column.

The mass composition of the product for feeding the evaporator at the bottom of the rectification column in the case of the production of butyl acrylate in the presence of phenothiazine, as the inhibitor mainly used, is as follows:
Butyl acrylate (40-50%)
Butyl hydroxypropionate (BHP): 0.5-1.5%
Butyl butoxypropionate (BBP): 50-70%
Butyl acryloxypropionate (AA/BuA): 2-3%
Dibutyl maleate: 1-3%
Phenothiazine: 1-3%.

The residence time in the evaporator is about one minute.

With reference to FIG. 1, which represents the preferred mode of the invention, the topping section comprises a distillation column having an equivalent of 10 and 30 theoretical plates, preferably 10 to 15 theoretical stages. The inserts used for the column may be valve trays or perforated weir trays, crossflow trays such as Dual Flow Trays, Ripple Trays, Turbo Grid Shells, or ordered packing, for instance structured packing such as Mellapack 250× from Sulzer.

The topping column is fed in the upper third of this column, preferably between the theoretical plates 3 to 10 counted from the top of the column. The top stream of the column essentially comprises the unreacted reactants. This upgradable stream is recycled to the reaction.

The column operates with a reflux ratio (flow rate of condensed liquid returned to the column/flow rate recycled to the reaction) of between 4/1 to 1/1, preferably 3/1. Advantageously, from 50 to 5000 ppm of polymerization inhibitor are introduced into the purification system according to the process of the invention.

Mention may be made, as polymerization inhibitors which can be used, of for example phenothiazine (PTZ), hydroquinone (HQ), hydroquinone monomethyl ether (HQME), di-tert-butyl para-cresol (BHT), para-phenylenediamine, TEMPO (2,2,6,6-tetramethyl-1-piperidinyloxy), di-tert-butylcatechol, or TEMPO derivatives such as OH-TEMPO, alone or mixtures thereof in all proportions, at contents in the reaction medium which may be between 50 ppm and 5000 ppm, optionally in the presence of depleted air, but generally in contents of between 150 ppm and 1000 ppm. Polymerization inhibitors can be added at different locations, with the introduction of reactants or at the top of the distillation column.

To make the inhibitors more effective, it is appropriate to inject oxygen, air or air so-called depleted air with 7% O₂ at the bottom of the column. Preferably, the amount of oxygen injected corresponds to a content of 0.2% to 0.5% relative to the amount of organic vapor in the column.

The column can operate under vacuum to minimize thermal exposure of heat-sensitive compounds within the column. Advantageously, the topping column operates under a vacuum ranging from 1000 Pa to 30 000 Pa.

The bottom stream preferably feeds the column, making it possible to obtain the purified ester at the bottom of the column between theoretical plate 6 to 9.

The pure product distillation column comprises an equivalent of 2 and 15 theoretical plates, preferably 6 to 12 theoretical stages. The inserts used for the column may be valve trays or perforated weir trays, crossflow trays such as Dual Flow Trays, Ripple Trays, Turbo Grid Shells, or ordered packing, for instance structured packing such as Mellapack 250× from Sulzer.

The column top stream consists of high-purity butyl acrylate with an ester purity specification greater than 99.5%.

The column operates with a reflux ratio (flow rate of condensed liquid returned to the column/flow rate of pure product) of between 1/8 to 1/1, preferably 1/4. Like the topping column, it is stabilized and air or depleted air (7% O₂) is injected at the bottom of the column. The column can operate under vacuum to minimize thermal exposure of heat-sensitive compounds within the column. Advantageously, the pure product column operates under a vacuum ranging from 1000 pascal to 20 000 pascal.

Advantageously, the operating temperature is between 50° C. and 160° C.

The bottom stream is concentrated on a thin-film evaporator, suitable for viscous, fouling products and soiled liquids. Other technologies such as grille plate exchangers do not meet this need because the residence time therein is too long.

The evaporation is carried out in a temperature range of 80° C. to 100° C. and more especially between 90° C. and 100° C., in a pressure range of between 800 Pa and 2000 Pa.

According to one embodiment, the polymerization inhibitor content in the bottom stream of the rectification column containing the Michael adduct stream is less than 3% in this bottom stream.

The top adduct stream can then feed a cracker as described in FR 2901272. This cracker is a forced recirculation reactor comprising an external exchanger. The temperature of the reaction medium is between 160° and 180° C. The pressure in this reactor is maintained at atmospheric pressure. The bottom product constitutes the ultimate residue and is sent to the appropriate channel. The top product condensed at a temperature of 20° C. to 30° C. is sent to the reactor. It is not necessary to inject air or depleted air into this reactor because the residual content of stabilizer in this adduct stream is sufficient to avoid problems of solid formation.

The examples below illustrate the present invention without, however, limiting the scope thereof.

### Experimental Section

In the examples, the percentages are shown by weight, unless otherwise indicated, and the following abbreviations were used:
PTZ: Phenothiazine
BuA: butyl acrylate
BuOH: butanol
BBP: butyl butoxypropionate
DBE: dibutyl ether
BAP: Butyl acryloxypropionate

### Example 1: Test with a Thin-Film Evaporator

A commercially available 400 kPa steam-heated DV210/1 m² thin-film evaporator, operating at a pressure of 130 Pa, is fed at a rate of 50 kg/h with a mixture comprising: BuA: 9%; BuOH: 3.5%; BBP: 67%; BAP: 5.7%; PTZ: 1.8%; the remainder to 100%: heavy residue.

The top product (40 kg/h) is condensed at 20° C. in a 4 m² tubular exchanger. It is stabilized by adding a solution of 250 g/h butyl acrylate containing 2% phenothiazine. This top product comprises: 12.5% BuA; 75% BBP; 3% butanol and 900 ppm phenothiazine. The evaporator bottom product containing PTZ (6%) does not have particles. After one week of operation, the exchanger and circuits are clean.

### Example 2: Test with a Plate and Shell Evaporator

A 400 kPa steam-heated Vahterus plate and shell evaporator type 1 PSHE4/2HA-40/1/1 1.2 m² sold by Kapp, operating at a pressure of 130 Pa, is fed at a rate of 50 kg/h with a mixture comprising: BuA: 9%; BuOH: 3.5%; BBP: 67%; BAP: 5.7%; PTZ: 1.8%; the remainder to 100%: heavy residue. The residence time of the liquid phase in the exchanger is 20 min.

The evaporator bottom product containing PTZ (6%) does not have particles. However, after two days of operation, the exchanger is clogged.

### Example 3: Process According to the Invention

The simulated evaporation process under Aspen V10 is shown in appended FIG. 2. Table 1 below indicates the working conditions used and the compositions of the various phases.

This simulation shows, on the one hand, that the evaporator under these conditions generates a gas phase in which the inhibitor is absent and, on the other hand, that it is possible to obtain a liquid adduct stream and a butyl acrylate stream which can be sent to the cracker and to the column feed.

TABLE 1

| | | | Column | | |
|---|---|---|---|---|---|
| | | Evapo- | return | | |
| | | rator | liquid | | Evaporator |
| | Units | feed | phase | Adducts | bottom |
| Phase | | Liquid | Liquid | Liquid | Liquid |
| Temper- | ° C. | 109.6 | 20 | 68.5 | 95 |
| ature | | | | | |
| Pressure | Pas- | 17 330 | 100 000 | 1000 | 1000 |
| | cals | | | | |
| Mass flow | Kg/h | 14 000 | 5880 | 6529 | 1591 |
| rate | | | | | |
| Mass | | | | | |
| fraction | | | | | |
| BuA | % | 43 | 89 | 11 | 2 |
| BuOH | % | 0 | 0 | 0 | 0 |
| DBE | ppm | 28 | 61 | 5 | 1 |
| PTZ | ppm | 12 000 | 0.3 | 700 | 102 300 |
| BBP | % | 56 | 10.9 | 8.9 | 8.7 |

1. A process for producing butyl acrylate by a reaction of direct esterification of acrylic acid with butanol in the presence of sulfuric acid as catalyst and of at least one polymerization inhibitor, resulting in production of a crude reaction mixture containing butyl acrylate, residual acrylic acid and residual butanol, butyl hydrogen sulfate, traces of sulfuric acid and impurities resulting from side reactions, said process comprising neutralization and washing steps with water leading to the production of a reaction mixture free of acidic impurities, wherein said reaction mixture washed of acidic impurities is subjected at least to the following steps:
i) topping in a distillation column to obtain:
at the top, a stream composed essentially of unreacted reactants;
at the bottom, a stream comprising the desired ester and heavy by-products;
ii) subjecting the bottom stream from the topping column to a rectification column to separate:
at the top, purified butyl acrylate;
at the bottom, a stream containing heavy by-products, which is concentrated on a thin-film evaporator generating three streams:
a butyl acrylate stream to be recycled to the rectification column;
a Michael adduct stream for feeding the cracker;
a stream containing residue containing polymerization inhibitors;
iii) sending the stream containing Michael adducts to a thermal and catalytic cracker to obtain:
at the top, the noble products resulting from the cracking which are recycled to the reaction;
at the bottom, the ultimate residue that is sent to a waste treatment plant.

2. The process as claimed in claim 1, wherein said acidic impurities are sulfuric acid, butyl hydrogen sulfate, acrylic acid dimer and residual acrylic acid.

3. The process as claimed in claim 1, wherein the polymerization inhibitor is chosen from the group selected from: phenothiazine (PTZ), hydroquinone (HQ), hydroquinone monomethyl ether (HQME), di-tert-butyl para-cresol (BHT), para-phenylenediamine, TEMPO (2,2,6,6-tetramethyl-1-piperidinyloxy), di-tert-butylcatechol, OH-TEMPO, and mixtures thereof in all proportions.

4. The process as claimed in claim 1, wherein the polymerization inhibitor is used in the reaction or in purification steps at contents between 50 ppm and 5000 ppm.

5. The process as claimed in claim 1, wherein the polymerization inhibitor is added at different locations, with the reactants or at the top of the distillation column.

6. The process as claimed in claim 1, wherein the evaporation is carried out in a temperature range of 80° C. to 100° C., and at a pressure of between 800 Pa and 2000 Pa.

7. The process as claimed in claim 1, wherein the top stream from the evaporator is cooled in two successive steps:
a. a partial condensation in a temperature range of from 50 to 80° C. at the same pressure as that of the evaporator to obtain, on the one hand, a liquid stream of Michael adducts, having a content of inhibitors of less than 1000 ppm, going to feed the cracker, and, on the other hand, a vapor stream, and
b. a condensation of this vapor stream in a temperature range of from 20° C. to 40° C. before taking up this liquid stream via a pump and feeding the rectification column.

8. The process as claimed in claim 1, wherein the cracking is carried out at a temperature of 160° C. to 180° C. at atmospheric pressure.

9. The process as claimed in claim 1, wherein the butyl acrylate obtained has a purity greater than 99.5%.

10. The process as claimed in claim 1, wherein the polymerization inhibitor content in the bottom stream of the rectification column containing the Michael adduct stream is less than 3%.
